(19) United States
(12) Patent Application Publication
(10) Pub. No.: US 2021/0112836 A1
(43) Pub. Date: Apr. 22, 2021

(54) INTESTINAL FLORA-IMPROVING HEALTH FOOD

(71) Applicant: MUROTO KAIYOUSHINNSOUSUI CO., LTD, Kochi (JP)

(72) Inventors: Shizuo KOMATSU, Kochi (JP); Hiroaki TAKEUCHI, Kochi (JP)

(21) Appl. No.: 16/981,899

(22) PCT Filed: Feb. 18, 2019
(86) PCT No.: PCT/JP2019/005915

§ 371 Date: Sep. 17, 2020

(30) Foreign Application Priority Data
Mar. 19, 2018 (JP) 2018051567

(51) Int. Cl. A23L 11/00; A61K 35/741; A61K 33/06; A23L 33/16; A61K 36/48; A23L 33/105; A61P 1/14

(52) U.S. Cl. CPC A23L 11/00; A61K 35/741; A61K 33/06; A61P 1/14; A61K 36/48; A23L 33/105; A23L 33/16

(57) ABSTRACT

The present invention provides intestinal flora-improving health food which improves intestinal flora by containing bittern to increase and activate Equol-producing bacteria. The intestinal flora-improving health food comprises 0.8 wt % to 1.5 wt % of bittern relative to the total weight of the health food, wherein the bittern per 100 g comprises 2500 mg to 6000 mg of magnesium content and 500 mg to 2000 mg of calcium content; wherein the proportion of magnesium content to calcium content in the bittern is 2 to 7, and wherein the hardness of the bittern is at 144000 mg/L to 383000 mg/L.

### BACKGROUND OF THE INVENTION

### Field of the Invention

The present invention relates to intestinal flora-improving health food.

### Description of Related Art

Many types of bacteria live in intestines of animals including human being and these intestinal bacteria form a type of ecosystem called intestinal flora (or intestinal bacterial flora).

Intestinal bacteria grow by metabolizing foods ingested and fed into the intestines by a host, but metabolites generated by the metabolism vary depending on the type of the bacteria. Among these bacteria, there are good bacteria whose metabolites contribute to maintaining the health of the host, bad bacteria whose metabolites harm the health of the host, and opportunistic bacteria which could become beneficial or harmful depending on the intestinal flora balance.

The ideal balance of the intestinal bacteria in the intestinal flora is said to be; good bacteria:bad bacteria:opporunistic bacteria=2:1:7. However, the balance of the intestinal bacteria in the intestinal flora is not constant but varies depending on the individual differences in hosts. Also, it is known that the intestinal flora balance changes due to changes in the host's dietary environment and physical conditions, medication, stress and so on.

It is reported that increase in bad bacteria resulting in deterioration of intestinal flora balance (imbalancing) could cause poor health, such as skin problems or stiffed shoulders induced by the detrimental metabolites absorbed through intestinal walls into bloodstream, and lifestyle-related disease, as well as digestive system disorders, such as constipation or diarrhea.

On the other hand, intestinal flora-improving foods conventionally include lactic acid-fermented foods containing lactic acid bacteria that are good bacteria, oligosaccharide, and so on.

Improvement of intestinal flora will help the bowels work better. Also, good bacteria are beneficial to hosts, as they produce beneficial metabolites (vitamins, for example).

As a method of examining the intestinal flora balance, a method of identifying intestinal bacteria in sampled feces is generally utilized.

Another method of examining the intestinal flora balance may include a method of examining the host's blood, in consideration that metabolites of the intestinal bacteria are absorbed in bloodstream.

Also, the above-mentioned poor health allows for anticipation of deteriorating intestinal flora balance, although it is not an accurate examination method.

Now, according to the Japanese Patent No. 5502449, the food with royal jelly fermented with lactic acid bacteria is well known as food having the effect of improving the intestinal flora balance.

Also, according to the Japanese Patent No. 4693390, “functional water” is well-known, which comprises indigestible carbohydrates and bittern, and has the intestinal flora-improving effect.

### BRIEF SUMMARY OF THE INVENTION

### Problems to be Solved by the Invention

By the way, the food disclosed in the Japanese Patent No. 5502449 is described to increase the total number and the ratio of good bacteria (lactic acid bacteria) while decreasing the total number and the ratio of bad bacteria and Escherichia coli bacteria. However, the food in the Japanese Patent No. 5502449 refers to food fermented with lactic acid bacteria. Fermented foods are difficult to produce and store, as they are required to promote proliferation of only the targeted beneficial bacteria while suppressing proliferation of germs and destructive bacteria.

The functional water in the Japanese Patent No. 4693390 is disclosed to contain bittern and contribute to improvement of the intestinal flora, however, in that disclosure, the bittern is added to milden the water in mouth and improve its taste, and it is oligosaccharide that contributes to the improvement of the intestinal flora.

Now, the inventors of the present invention found that the intestinal flora is improved by ingesting bittern in specific composition. Then, after a number of contemplations, the inventors of the present invention have come to invent intestinal flora-improving health food with the aid of bittern in composition effective to improve the intestinal flora.

Based on the finding that ingestion of bittern in specific composition above improves the intestinal flora, the present invention is directed to provide intestinal flora-improving health food which improves intestinal flora by containing such bittern.

### Means for Solving the Problems

The invention in the first aspect relates to intestinal flora-improving health food comprising 0.8 wt % to 1.5 wt % of bittern relative to the total weight of the health food, wherein the bittern per 100 g comprises 2500 mg to 6000 mg of magnesium content and 500 mg to 2000 mg of calcium content; wherein the proportion of magnesium content to calcium content in the bittern is 2 to 7, and wherein the hardness of the bittern is at 144000 mg/L to 383000 mg/L.

The invention in the second aspect relates to intestinal flora-improving health food in the first aspect, wherein the intestinal flora-improving health food is tofu.

The invention in the third aspect relates to intestinal flora-improving health food in the first aspect, wherein the intestinal flora-improving health food is fermented soybeans (natto).

The invention in the fourth aspect relates to intestinal flora-improving health food in the first aspect, wherein the intestinal flora-improving health food is fermented bean paste (miso).

The invention in the fifth aspect relates to intestinal flora-improving health food comprising 90.0 wt % to 99.0 wt % of bittern relative to the total weight of the health food, wherein the bittern per 100 g comprises 2500 mg to 6000 mg of magnesium content and 500 mg to 2000 mg of calcium content; wherein the proportion of magnesium content to calcium content in the bittern is 2 to 7, and wherein the hardness of the bittern is at 144000 mg/L to 383000 mg/L, and wherein the intestinal flora-improving health food is a supplement contained in a capsule.

### Effect of the Invention

According to the invention in the first aspect, the intestinal flora-improving health food comprises 0.8 wt % to 1.5 wt % of bittern relative to the total weight of the health food, wherein the bittern per 100 g comprises 2500 mg to 6000 mg of magnesium content and 500 mg to 2000 mg of calcium content; wherein the proportion of magnesium content to calcium content in the bittern is 2 to 7, and wherein the hardness of the bittern is at 144000 mg/L to 383000 mg/L. Thereby, it is possible to provide intestinal flora-improving health food which improves intestinal flora by containing bittern.

According to the invention in the second aspect, the intestinal flora-improving health food is tofu. Bittern is used in producing tofu. Therefore, it is possible to obtain intestinal flora-improving health food which improves the intestinal flora, by composing the bittern in composition above.

Equol-producing bacteria, which are a type of good bacteria, grow (i.e., proliferate) by metabolizing isoflavones, especially daidzein, and producing Equol. Now, as lots of isoflavones, such as daidzein, are contained in soybeans which is a basic ingredient of tofu, ingesting soy ingredients together with the bittern in composition above can increase and activate the Equol-producing bacteria more efficiently and improve the intestinal flora.

According to the invention in the third aspect, the intestinal flora-improving health food is fermented soybeans (natto). Same as tofu, fermented soybeans (natto) are also food produced from soybeans as its basic ingredient. Therefore, in this configuration too, ingesting soy ingredients together with the bittern in composition above can increase and activate the Equol-producing bacteria more efficiently and improve the intestinal flora.

According to the invention in the fourth aspect, the intestinal flora-improving health food is fermented bean paste (miso). Same as tofu, fermented bean paste (miso) is also food produced from soybeans as its basic ingredient. Therefore, in this configuration too, ingesting soy ingredients together with the bittern in composition above can increase and activate the Equol-producing bacteria more efficiently and improve the intestinal flora.

According to the invention in the fifth aspect, as the intestinal flora-improving health food is in the form of a supplement contained in a capsule, it can be easily ingested without bitterness of the bittern, even if the amount of bittern is increased. Also, it is possible to ingest intestinal flora-improving health food in addition to regular meals to improve intestinal flora.

### DETAILED DESCRIPTION OF THE INVENTION

Hereinafter, the intestinal flora-improving health food of the present invention is described in detail.

Many bacteria live in human intestines and so on. In intestines of one person, 100 to 3000 kinds of bacteria grow, or equivalently, 100 trillion to 1 quadrillion intestinal bacteria live there.

Intestinal flora refers to a biocenosis (a kind of ecosystem) comprised of these intestinal bacteria. The intestinal flora balance changes due to changes in the host's dietary environment and physical conditions, medication, stress and so on. Among intestinal bacteria, there are good bacteria which contribute to maintaining the health of the host, bad bacteria which harm the health of the host, and opportunistic bacteria which could become beneficial or harmful depending on the intestinal flora balance, and so on.

Bacteria in Bifidobacterium and in Lactobacillus are typical of good bacteria, and most of them are the bacteria also called lactic acid bacteria. Bacteria in Clostridium and in Staphylococcus are typical of bad bacteria.

A method of examining the intestinal flora balance may include a method of measuring lactic acid bacteria, good bacteria, and bacteria in Clostridium, bad bacteria, from the sampled feces, in a culture medium for bacteria measurement (the Japanese Patent No. 5502449), or a method of checking the subject's bowel movements and feces state by questionnaires (the Japanese Patent No. 4693390).

Since the metabolites of intestinal bacteria are absorbed through the host's intestinal wall into the bloodstream, it is also possible to examine the intestinal flora balance by a blood test. Also, since a part of these metabolites are discharged in urine, it is also possible to examine the intestinal flora balance by a urine test.

It is ideal that intestinal flora maintains the balance of intestinal bacteria as good bacteria:bad bacteria:opportunistic bacteria=2:1:7, however in general, bad bacteria tend to dominate in the intestinal flora. Therefore, it is preferable to increase good bacteria.

Now, Equol-producing bacteria also exist as good bacteria. Equol-producing bacteria metabolize the isoflavone (especially daidzein) in soybeans etc., and produce Equol.

It has been reported that increase in Equol in bloodstream due to increase in Equol-producing bacteria could present various epidemiologically beneficial effects to the host (Kazuhiro HIRAYAMA “Metabolism of Soy Isoflavone by Intestinal Flora”. Journal of intestinal microbiology, Volume 19 (2005), Issue 1, p. 17-23; Yoshiko ISHIMI, Yuko TOUSEN “Chounaisaikin ga tsukuridasu Daizuisofurabontaishasanbutsu no Yuuyousei to Anzensei Ekuooru no Kanousei” (Effects and safety of soybean isoflavone metabolites produced by intestinal bacteria Possibility of Equol)”. KAGAKU TO SEIBUTSU, Volume 51, Issue 2 (2013), p. 74-77; Tomomi Ueno et al. “Distribution of 24-h urinary equol excretion as an indicator of the physiological range in healthy Japanese equol excretors”. Journal of Functional Foods, Volume 7, March 2014, P. 129-135).

Also, it is known that a part of Equol is discharged as urine of the host. Kazuhiro HIRAYAMA “Metabolism of Soy Isoflavone by Intestinal Flora”. Journal of intestinal microbiology, Volume 19 (2005), Issue 1, p. 17-23 discloses that when an germfree animal without intestinal flora was fed with soybean foods, daidzein was found in its urine, but Equol was not found, and it is also known that the amount of Equol to be produced by the Equol producing bacteria in the host's intestines is positively correlated with the amount of Equol in the host's urine.

In the present invention, intestinal flora improvement is to be evaluated by increase in and activation of Equol-producing bacteria, good bacteria, as index. Also, increase in and activation of Equol-producing bacteria are evaluated by the Equol content in the host's urine

Here, Equol-producing bacteria are general terms for the bacteria which metabolize isoflavones (especially daidzein) and produce Equol.

Equol-producing bacteria include Adlercreutzia equolifaciens, Asaccharobacter celatus AHU1763, Asaccharobacter celatus gen, nov. sp. nov strain do03T, Bacteroides ovatus, Bifidobacterium animalis, Coriobacteriaceae sp MT1B9, Eggerthella sp (YY7918, Julong732, MT4s-5), Enterococcus faecium, Eubacterium sp D1 and D2, Finegoldia magna, Lactobacillus mucosae, Lactobacillus sp Niu-O16, Lactococcus garvieae (Lc 20-92), Ruminococcus productus, Slackia sp (HE8, FJK1, TM-30, NATTS) Slackia equolifaciens (Strain DZE), Streptococcus intermedius, Veillonella sp, Mixture of Lactobacillus mucosae EPC4, Corynebacteriales sp E-17, and so on (Kenneth D. R. Setchell et al. “Equol: History, Chemistry, and Formation”. THE JOURNAL OF NUTRITION, Volume 140, Issue 7, 1 Jul. 2010, p. 1355S-1362S).

Although isoflavone, in a precise sense, is a term which refers to 3-phenyl chromone shown by a below chemical formula (Formula 1), derivatives of isoflavone are also broadly referred to as isoflavones. In the present invention too, the derivatives of isoflavone are also called isoflavones.

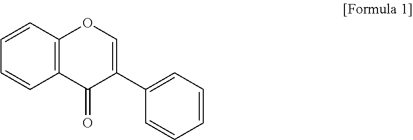 [SMILES: O=c1c(-c2ccccc2)coc2ccccc12]

Although there are many kinds of isoflavones, Glycine max (called soybeans hereinafter), in particular, includes lots of isoflavones, such as daidzein shown by a below chemical formula (Formula 2) and genistein shown by a below chemical formula (Formula 3).

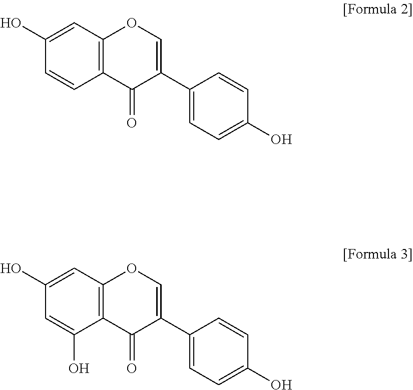 [SMILES: O=c1c(-c2ccc(O)cc2)coc2cc(O)ccc12 ; O=c1c(-c2ccc(O)cc2)coc2cc(O)cc(O)c12]

Also, these isoflavones have antioxidative capacity and female hormone, estrogenic effects, and thus, are shown to be effective to prevent and/or improve breast cancer, prostate cancer, osteoporosis, hypercholesterolemia, cardiac disease, and climacteric disorder.

Equol is a metabolite produced by the metabolism of Equol-producing bacteria, and is a substance shown by a below chemical formula (Formula 4).

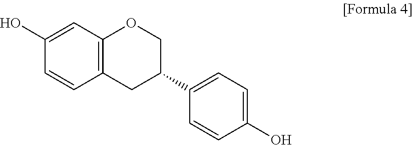 [SMILES: Oc1ccc([C@H]2COc3cc(O)ccc3C2)cc1]

Equol also has antioxidative capacity and female hormones, estrogenic effects, however, if compared with other isoflavones, Equol has higher antioxidative capacity and higher estrogenic effects, and maintains higher concentration in bloodstream for a longer period.

Therefore, compared with other isoflavones, Equol is more effective to prevent and/or improve breast cancer, prostate cancer, osteoporosis, hypercholesterolemia, cardiac disease, climacteric disorder, and so on.

The intestinal flora-improving health food in the present invention comprises bittern, wherein the bittern per 100 g comprises 2500 mg to 6000 mg magnesium content and 500 mg to 2000 mg calcium content; wherein the proportion of magnesium content to calcium content in the bittern is 2 to 7, and wherein the hardness of the bittern is at 144000 mg/L to 383000 mg/L.

By ingesting the intestinal flora-improving health food containing the bittern in composition above, it is possible to improve the intestinal flora.

The hardness of bittern can be obtained by the below equation 1. Hardness in general is calculated per L, but when calculated per 100 g, it is necessary to multiply 10 together with the specific gravity of bittern relative to water, so that conversion per 1 L will be available.

Hardness [mg/L]=((calcium content [mg/100 g]×2.5)+(magnesium content [mg/100 g]×4.1))×10×specific gravity (Equation 1)

In one embodiment, intestinal flora-improving health food may be provided as, but not limited to, processed foods or ready-made meals.

Also, in another embodiment, intestinal flora-improving health food may be used as, not limited to, supplements, such as in capsules containing intestinal flora-improving health food. Any method of using the intestinal flora-improving health food as a supplement can be utilized as long as it is obvious and well-known to person in the art.

In case the intestinal flora-improving health food in the present invention is provided as processed foods or ready-made meals, 0.8 wt % to 1.5 wt % of bittern maybe contained relative to the total weight of the health food. More preferably, 1.0 wt % to 1.5 wt % of bittern maybe contained relative to the total weight of the health food. In case the present invention is provided as a supplement, 90.0 wt % to 99.0 wt % of bittern maybe contained relative to the total weight of the health food.

In case the intestinal flora-improving health food is provided as processed foods or ready-made meals, the bittern content less than 0.8 wt % relative to the total weight of the health food is not sufficient for intestinal flora improvement, and the bittern content more than 1.5 wt % intensifies bitterness, thus, neither is preferable. 1.0 wt % to 1.5 wt % of bittern content is preferable, as it gives more effective effects on intestinal flora improvement while suppressing the bitterness.

In case the intestinal flora-improving health food is provided as a supplement, users can easily ingest it without feeling bitterness. It is possible to ingest the amount of bittern necessary to improve the intestinal flora, by increasing the bittern content relative to the total weight of the intestinal flora-improving health food, and making it into a generic capsule of 0.13 ml to 1.37 ml. Thereby, it is possible to ingest the intestinal flora-improving health food in addition to regular meals to improve intestinal flora.

If preferred, it is possible to use, but not limited to, bittern produced by condensing seawater by reverse osmosis membrane methods.

Deep-ocean water is preferably used to produce the bittern. Deep-ocean water is seawater at 200 m depth or below, and it is less affected by other waters, such as contaminated rivers, or less polluted by chemical substances. Also, as sunlight does not reach, the water temperature is low enough to contain propagation of microorganisms (germs). Therefore, applying the bittern derived from deep-ocean water to the present invention makes it possible to contain risks of negative effects on the intestinal flora balance by chemical substances or other negative effects by germs or germ-induced metabolites.

Appropriate amount of various additive agents can be blended in the present invention. The additive agents may include, but not limited to, excipients, disintegrating agents, binding agents, acidulants, foaming agents, artificial sweetening agent, flavoring agents, lubricants, colorants, stabilizers, pH adjusters, surfactants, and so on.

The excipients may include, but not limited to, starches, such as, corn starch, potato starch, wheat starch, rice starch, partially pregelatinized starch, pregelatinized starch, and porous starch; sugars, such as, lactose, sucrose, and glucose; sugar alcohol, such as, mannitol, xylitol, erythritol, sorbitol, and maltitol; and inorganic compounds, such as, magnesium aluminometasilicate, hydrotalcite, anhydrous calcium phosphate, precipitated calcium carbonate, calcium silicate, and light anhydrous silicic acid, and so on.

The binding agents may include, but not limited to, hydroxypropyl cellulose, hydroxypropyl methylcellulose, polyvinyl pyrrolidone, powdered gum arabic, gelatin, pullulan, and so on.

The disintegrating agents may include, but not limited to, starch, agar, carmellose calcium, carboxymethyl starch sodium, croscarmellose sodium, crospovidone, crystalline cellulose, F-MELT (Trademark, Fuji Chemical Industries Co., Ltd.), and so on.

The acidulants may include, but not limited to, citric acid, tartaric acid, malic acid, ascorbic acid, and so on.

The foaming agents may include, but not limited to, sodium bicarbonate, sodium carbonate, and so on.

The artificial sweetening agents may include, but not limited to, saccharin sodium, glycyrrhizin dipotassium, aspartame, stevia, thaumatin, and so on.

The flavoring agents may include, but not limited to, lemon oil, orange oil, menthol, and so on.

The lubricants may include, but not limited to, magnesium stearate, sucrose fatty acid ester, polyethylene glycols, talc, stearic acid, sodium stearyl fumarate, and so on.

The colorants may include, but not limited to, edible dye, such as Food Yellow No. 5, Food Red No. 2, and Food Blue No 2., edible lake dye, diiron trioxide, and so on.

The stabilizers may include, but not limited to, edetate sodium, tocopherol, cyclodextrin, and so on.

The pH adjusters may include, but not limited to, citrate, phosphate, carbonate, tartrate, fumarate, acetate, amino acid salt, and so on.

The surfactants may include, but not limited to, polysorbate 80, methyl cellulose, hydroxyethyl cellulose, sodium carboxymethyl cellulose, polyoxyethylene sorbitan monolaurate, gum arabic, powdered tragacanth, and so on.

Furthermore, the present invention may additionally comprise isoflavones, such as genistein and/or daidzein. As isoflavones are substances required for Equol-producing bacteria to grow, adding isoflavones can increase and activate Equol-producing bacteria more efficiently.

In connection with this, the amount of Equol produced also increases, leading to increase in Equol amount absorbed in bloodstream. Therefore, it can enhance the effectiveness to prevent and/or improve breast cancer, prostate cancer, osteoporosis, hypercholesterolemia, cardiac disease, climacteric disorder, and so on.

The present invention may be provided as soybean processed food.

As soybeans contain lots of isoflavones, providing soybean processed foods can increase and activate Equol producing bacteria in the user's intestines and improves the intestinal flora.

Soybean processed foods may include, for example, tofu, fermented soybeans (natto), fermented bean paste (miso), soy sauce, boiled beans, and so on.

Tofu products provided by the present invention may include, but not limited to, cotton tofu, soft cotton tofu, silken tofu, sealed silken tofu, oboro tofu (half-curdled tofu), adjusted cotton tofu, adjusted soft cotton tofu, adjusted silken tofu, adjusted sealed silken tofu, adjusted oboro tofu (adjusted half-curdled tofu), adjusted frozen tofu, processed frozen tofu, processed tofu, and so on.

Also, the present invention maybe provided as food further processed from tofu. In other words, it may be provided as grilled tofu, thin fried tofu, thick fried tofu, ganmodoki (fried tofu mixed with vegetables), koya tofu (freeze-dried tofu), dried tofu, and so on.

Fermented soybeans (natto) products provided by the present invention may include, for example, itohiki natto (standard fermented soybeans), shiokara natto (non-sticky fermented beans), dried natto, goto natto (further fermented natto), and so on.

Fermented bean paste (miso) products provided by the present invention may be divided into kome miso (rice miso), mugi miso (barley miso), mame miso (soybean miso), and mixed miso, depending on the type of Koji-mold to be used, but can be any of them. Also, it may be divided into red miso and white miso depending on how it is prepared, but both can be provided.

Soy sauce products provided by the present invention may include any of dark soy sauce, light soy sauce, tamari soy sauce (aged soy sauce), twice brewed soy sauce, white soy sauce, and so on.

### EXAMPLES

Hereinafter, preferable examples of the present invention are shown. However, the present invention should not be construed within the limitation of the examples.

### Example 1

### Preparation of Intestinal Flora-Improving Health Food

Bittern shown in the table 1 below was used.

TABLE 1

| Composition | Content |
|---|---|
| Magnesium (Mg) | 4090 mg/100 g |
| Calcium (Ca) | 1086 mg/100 g |
| Mg/Ca | 3.7 |
| Specific gravity | 1.24 |
| Hardness | 242473 mg/L |

1.2 wt % of the above bittern was added to organic soymilk in order to prepare a silken tofu. This organic soymilk per 100 g contained the ingredients shown in the table 2 below.

TABLE 2

| Composition | Content |
|---|---|
| Protein | 5.0 g |
| Fat | 3.0 g |
| Carbohydrate | 2.0 g |
| Sodium | 3.0 mg |
| Potassium | 217 mg |
| Calcium | 17 mg |
| Magnesium | 3.0 mg |
| Vitamin E | 0.2 mg |
| Lecithin | 205 mg |
| Isoflavone | 30 mg |

### Example 2

### A Comparison Test for Equol Amount in Urine with Ingestion of the Intestinal Flora-Improving Health Food

### (Test Methods)

Change in Equol amount in urine was compared among 11 adults (5 males and 6 females). The subjects were aged between 35 and 68 years old (51.91 years old at an average), and none of them took drugs, such as an antibacterial agent, and their blood pressures were in the normal range.

The subjects ingested 400 g of tofu in Example 1 per day for a month from Oct. 16, 2017 to Nov. 15, 2017 every day.

Before (Oct. 16, 2017), during (Oct. 31, 2017) and after (Nov. 15, 2017) the test, the subjects' urines were sampled and the amount of Equol in urine was measured with high-performance liquid chromatography (HPLC).

### (Result)

The amount of Equol in urine of the subjects before, during and after the test was compared. The result of the amount of Equol in urine of each subject before, during, and after the test is shown in the table 3 below. It is found that the amount of Equol in urine of the subjects increased, as the test progressed before, during and after the test.

This reveals that Equol-producing bacteria are increased and activated in the intestines of the subjects.

From the result above, it is found that the intestinal flora-improving health food in Example 1 improves the intestinal flora of the subjects, regardless of age or gender.

TABLE 3
Example 2 Test Result

| Subjects | | | Equol | | |
|---|---|---|---|---|---|
| Subject | | | (ng/μg · Cre) | | |
| NO. | Gender | Age | Before test | Duringtest | After test |
| 1 | M | 40 | 0.40 | 18.63 | 43.98 |
| 2 | M | 46 | 0.00 | 0.00 | 0.00 |
| 3 | M | 41 | 0.00 | 0.00 | 0.00 |
| 4 | M | 48 | 6.40 | 16.74 | 7.07 |
| 5 | M | 68 | 0.50 | 27.19 | 20.36 |
| 6 | F | 35 | 0.00 | 0.00 | 0.00 |
| 7 | F | 67 | 0.00 | 3.51 | 9.86 |
| 8 | F | 49 | 0.00 | 0.00 | 0.00 |
| 9 | F | 57 | 0.33 | 14.50 | 17.72 |
| 10 | F | 56 | 6.83 | 25.10 | 39.25 |
| 11 | F | 62 | 0.19 | 0.56 | 0.34 |

| | | | |
|---|---|---|---|
| Average | 2.09 | 15.17 | 19.80 |
| Standard deviation | 3.10 | 10.06 | 16.37 |
| Standard error | 1.17 | 3.80 | 6.19 |

### INDUSTRIAL APPLICABILITY

Based on the finding that ingestion of bittern in specific composition improves the intestinal flora, the present invention can provide intestinal flora-improving health food which improves intestinal flora by containing such bittern.

Furthermore, the improvement of intestinal flora by ingestion of intestinal flora-improving health food of the present invention increases and activates the Equol-producing bacteria, increasing the amount of Equol production, and resulting in increase in Equol content absorbed in bloodstream. Thereby, it is possible to obtain the effects to prevent and/or improve breast cancer, prostate cancer, osteoporosis, hypercholesterolemia, cardiac disease, climacteric disorder, and so on.

1. Intestinal flora-improving health food comprising 0.8 wt % to 1.5 wt % of bittern relative to the total weight of the health food,
wherein said bittern per 100 g comprises 2500 mg to 6000 mg of magnesium content and 500 mg to 2000 mg of calcium content;
wherein the proportion of magnesium content to calcium content in said bittern is 2 to 7, and
wherein the hardness of said bittern is at 144000 mg/L to 383000 mg/L.

2. The intestinal flora-improving health food in claim 1, wherein said intestinal flora-improving health food is tofu.

3. The intestinal flora-improving health food in claim 1, wherein said intestinal flora-improving health food is fermented soybeans (natto).

4. The intestinal flora-improving health food in claim 1, wherein said intestinal flora-improving health food is fermented bean paste (miso).

5. Intestinal flora-improving health food comprising 90.0 wt % to 99.0 wt % of bittern relative to the total weight of the health food,
wherein said bittern per 100 g comprises 2500 mg to 6000 mg of magnesium content and 500 mg to 2000 mg of calcium content;
wherein the proportion of magnesium content to calcium content in said bittern is 2 to 7; and
wherein the hardness of said bittern is at 144000 mg/L to 383000 mg/L, and,
wherein said intestinal flora-improving health food is a supplement contained in a capsule.
